(19) United States
(12) Patent Application Publication
(10) Pub. No.: US 2007/0198057 A1
(43) Pub. Date: Aug. 23, 2007

(54) Method and device for closing holes in tissue

(76) Inventors: Daniel Gelbart, (US); Samuel Victor Lichtenstein, (US)

Correspondence Address: Dan Gelbart, 4706 Drummond Dr., Vancouver, BC, V6T-1B4, CA

(21) Appl. No.: 11/357,011

(22) Filed: Feb. 21, 2006

(51) Int. Cl. A61B 17/08

(52) U.S. Cl. 606/213

(57) ABSTRACT

A device for closing holes tissue is delivered via a catheter to the inside of a body lumen such as a heart. An elastic barbed clip is expanded, pulled into the tissue and released, pulling the tissue with it. The operation is fully reversible.

### FIELD OF THE INVENTION

The present invention relates to surgery and in particular to closing holes in tissue during minimally invasive surgery. The invention is particularly useful for closing holes left by catheters during percutaneous surgical procedures such as minimally invasive cardiac surgery and other surgeries requiring access to body lumens.

### BACKGROUND OF THE INVENTION

More and more surgical procedures are performed percutaneously by the use of catheter-delivered devices. The main advantages are fast patient recovery and lower costs to the medical system. Some tissues, such as muscular tissue or arterial walls, do not seal well and are sometimes subject to blood pressure; therefore they require an immediate hemostatic seal after the surgery. Prior art solutions mainly rely on some form of a plug, such as an expanding foam plug, expanding metal plug or a barbed plug to seal the hole. The main disadvantage of plugs is that in order to form a good seal they are forcing the hole to become larger, rather than the more natural way which is to shrink the hole in order to promote healing. A prior art device operating by shrinking the hole is the Star Closure device sold by Abbott Vascular (www.abbottvasculardevices.com); however this device is only suitable to thin walled body lumens as it relies on folding the tissue. When sealing larger holes in thicker tissue the gripping points for pulling the tissue inwards have to be spread over an area significantly larger than the hole size, similar to what is done in traditional suturing. Attaching the closure device too close to the hole does not allow sufficient forces to be applied, therefore creating a marginal closure.

Another major shortcoming of the Star Closure and other devices is that the operation is not reversible. It is sometimes required to remove the closure, as in the case of bleeding or an additional procedure.

It is therefore desired to provide a hole closure method that provides an immediate liquid and gas tight closure and it can be delivered by a catheter to the inside wall of a body lumen.

It is also desired to provide a closure method suitable for a large range of tissue thicknesses and hole sizes.

It is also desired to be able to test, and if required to remove, the closure.

It further would be desired for the closing device to have permanent elastic properties to accommodate any movement or future changes in the tissue. Furthermore, the gripping area of the closure device has to be significantly larger than the original hole.

### SUMMARY OF THE INVENTION

In view of the foregoing, the invention provides a method and device for closing holes in body lumens, and in particular in the heart and blood vessels, achieving an immediate hemostatic seal. The device can be applied via a wide range of catheters sizes to close a wide range of round and elongated holes with performance and reliability of traditional sutures but without requiring access to the tissue, except via the catheter. Furthermore, the device can be removed via the same catheter, and by using the same tools, used to install it and can be re-used immediately if so desired. The device has a high degree of elastic compliance allowing a wide accommodation range to changes in the tissue.

These and other objects of the present invention are achieved by providing a flexible clip that is temporarily attaches to an insertion tool. The clip has three different position: a storage position, in which it is folded inside a delivery tube; and expanded position, in which it opens up to reach an area significantly larger than the hole, and a closed position in which elastic forces try to close the clip, pulling the tissue with it to close the hole. The clip has multiple sharp barbs for gripping the tissue and a stem for attaching to the insertion tool, as well as for re-attaching in case removal is required.

Methods for implanting and removal of the device are also provided.

The invention will become apparent by studying the drawings and the detailed description.

### BRIEF DESCRIPTION OF THE DRAWING

FIG. 1 is a perspective view of the invention and the installation tool.

FIG. 2-a to FIG. 2-f are sectional views showing the steps in installing the device using the installation tool.

FIG. 3 is a perspective “exploded” view of the device.

FIG. 4 is a perspective “exploded” view of an alternate embodiment.

FIG. 5 is an “exploded” view of another alternate embodiment.

FIG. 6-a is a perspective view of an alternate embodiment in the relaxed state.

FIG. 6-b is a perspective view of the same alternate embodiment in the expanded state.

FIG. 7-a is a sectional view of the tool used to install the device embodiment of FIG. 6-a in the relaxed state.

FIG. 7-b is a sectional view of the tool used to install the device embodiment of FIG. 6-a in the expanded state.

FIG. 8-a to FIG. 8-f are sectional views showing the steps in removing the device.

### DETAILED DESCRIPTION OF THE INVENTION

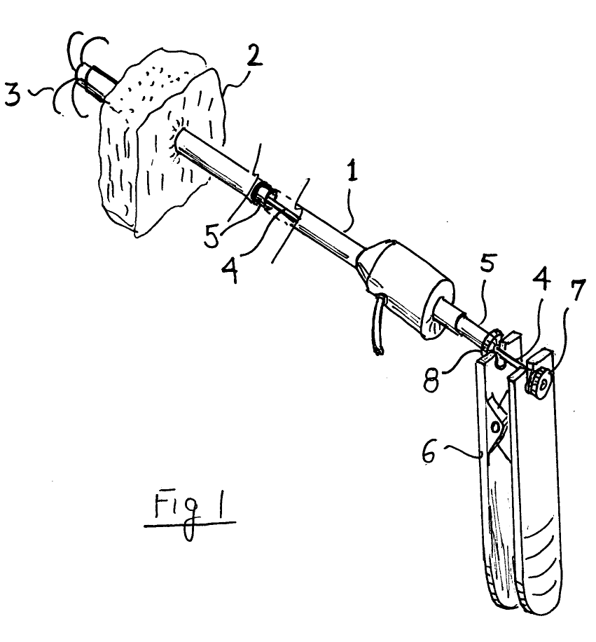

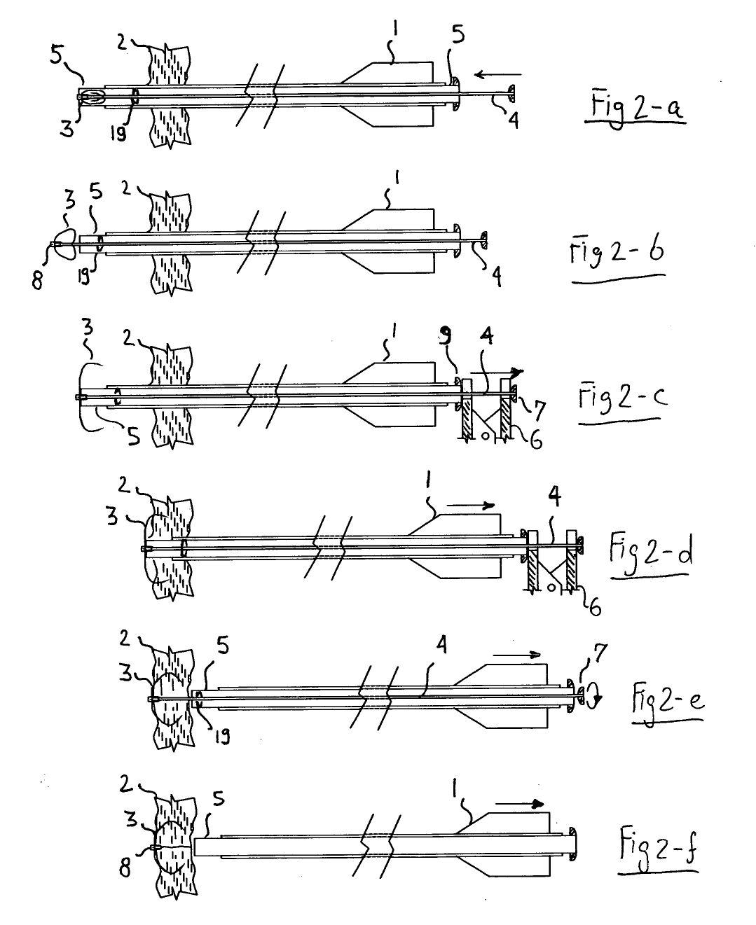

Referring to FIG. 1, a hole closure clip 3 is inserted into a body lumen such a cavity in the heart via catheter 1. Catheter 1 has a seal allowing insertion and removal of tools without much blood loss. This is well known in the art of minimally invasive surgery. When the surgical procedure is completed and hole needs to be closed, tube 5 carrying clip 3 mounted on rod 4 is inserted via catheter 1 through the tissue 2. Both rod 4 and tube 5 have flanges 7 and 8 allowing a pulling tool 6 to exert a significant pulling force on rod 4 relative to tube 5. Pulling tool 6 may be made of plastic or metal, plastic being preferred if tool is to be disposable. Rod 4 and tube 5 are preferable made of stainless steel and closure device 3 is made of Nitinol, a highly flexible Nickel-Titanium alloy well known in the art of medical devices. Tool 5 is similar in construction to the well known clothe-pin. Since the elastic range of Nitinol is about ten times larger than steel, the clip 3 can be made to fold into a small diameter tube and expand to grip the tissue over an area significantly larger than the area of the hole, in order to establish reliable closure. When clip 3 is released it tries to return to its natural (relaxed) shape, which covers a significantly smaller area, pulling the tissue with it and forming an instant hemostatic seal. These steps are shown in FIG. 2-a to FIG. 2-f.

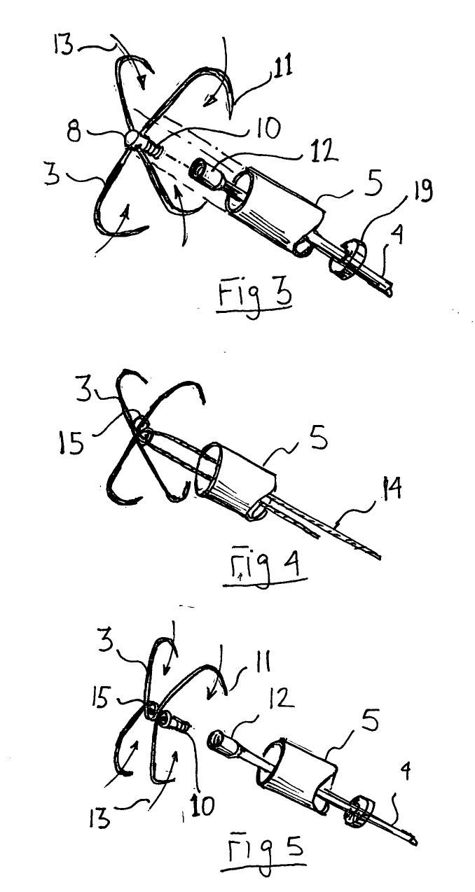

In FIG. 2-a the tube 5 containing the folded clip 3 mounted on rod 4 is inseterd via catheter 1 through the wall of the tissue 2. Rod 4 is pushed forward by finger pressure till it is felt that clip 3 is released from tube 5 (or moved till it reaches a pre-determined distance), as shown in FIG. 2-b. At this pint it is pulled back and pulling tool 6 is installed by sliding it on rod 4. Pulling tool 6 can be permanently mounted on rod 4 or slide in and out via two slots as shown in FIG 1. The slots rest against flanges 7 and 8. Flange 7 is rigidly connected to rod 4 while flange 8 is rigidly connected to tube 5. Using pulling tool 6, rod 4 is pulled out a pre-determined amount which forces clip 3 to open as shown in FIG. 2-c, as it rests against end of tube 5. At this point the whole assembly, including catheter 1, is pulled back to engage the sharp barbs of tool 3 in tissue 2. This is shown in FIG. 2-d. An enlarged view of clip 3 is shown in FIG. 3. In FIG. 3, clip 3 comprises of multiple sharp barbs 11 held by a threaded stem 8. Clip 3, including barbs 11 are made of Nitinol wire typically 0.6-0.8 mm in diameter, Stem 8 can be made of type 316 stainless steel and held to wires by crimping. It contains a threaded portion 10 for attaching to rod 4. The end of rod 4 has a mating thread 12. When clip 3 rests on edge of tube 5 it can be opened widely by pulling rod 4 and barbs 11 can reach over an area having a diameter from 1.5 to over 3 times the diameter of the hole. This is important to achieve proper hemostatic closure.

When rod 4 is detached from clip 3, the natural elasticity pulls barb 11 in the direction shown by arrows 13 and the tissue is pulled with them. Centering ferrule 19 on rod 4 keeps the location of clip 3 centered to tube 5, therefore centered to hole in tissue.

Returning now to FIG. 2-e, pulling tool 6 is released and removed allowing clip 3 to compress the tissue. Rod 4 is removed by turning flange 7 to unthread rod. After rod is removed the closure can be tested for leaks by leaving tube 5 in place. When used in the heart, any imperfection in closure will cause blood to come out of tube 5. In such a case the clip 3 can be removed and re-installed as shown later on in this disclosure. One verified, both tube 5 and catheter 1 are removed.

FIG. 4 shows an alternate design for clip 3. The main differences are that the Nitinol wire is bent into a loop 15 to add elasticity and a string 14 is used as a method of holding clip 3 to tube 5. The string can be removed by releasing one end.

FIG. 5 shows another alternate design, preferred when hole is an elongated cut rather than a round hole. Clip 3 is bent to have barbs 11 move in parallel rather than radially, as shown by arrows 13. Clip 3 is placed with the direction of motion 11 perpendicular to long dimension of hole in tissue. Loops 15 are used to add elasticity, as in FIG. 4.

FIG. 6 shows yet another alternate design. The clip 3 can be fabricated from Nitinol sheet, tubing or wire. Preferred way would be laser-cut tubing. FIG. 6-a shows the clip in the relaxed state, FIG. 6-b shows it in the expanded state. This design is suitable when a large number of barbs 11 are desired or for thin-walled lumens.

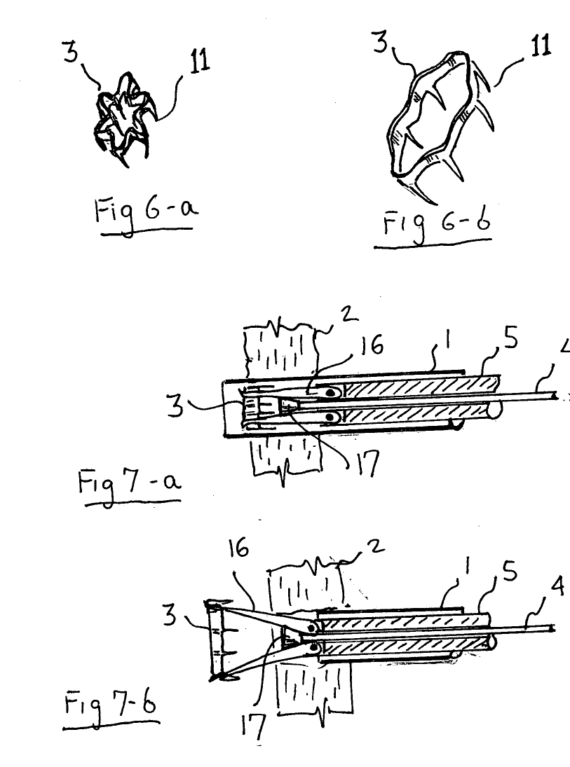

The tool used to expand the clip is shown in FIG. 7-a (relaxed state) and FIG. 7-b (expanded state). Rod 4 is equipped with a tapered end 17 used to expand four pivoting arms 16. The sequence of operations is identical to the sequence shown in FIG. 2-a to FIG. 2-f.

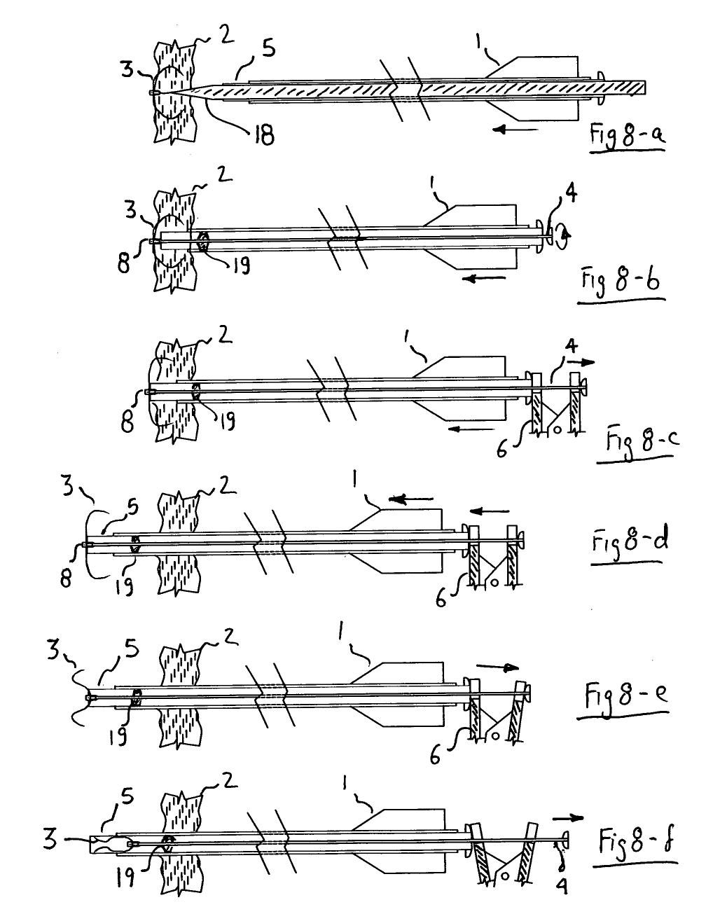

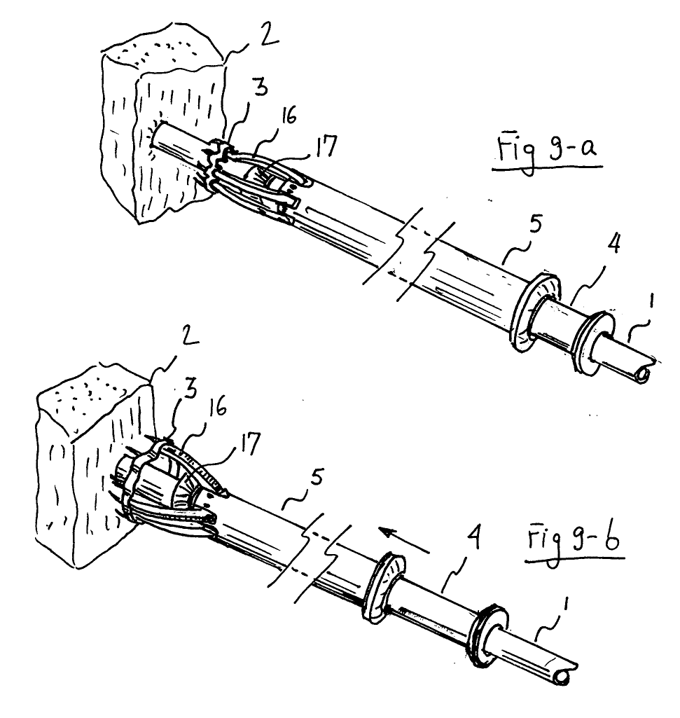

It is desirable to be able to reverse the clip installation and, if needed, remove the clip completely via the same catheter used to install it. The current invention, in all its forms, allows this to be done. Referring now to FIG. 8-a to FIG. 8-f, the sequence of partial and full removal is shown.

In FIG. 8-a a dilator 18 is used to expand the opening in the tissue 2 as well as the surrounding tissue, in order to feed tube 5 back into its original position. In FIG. 8-b Rod 4 is inserted in tube 5 and is attached to clip 3 by threading it onto stem 8 of clip 3. Centering ferrule 19 keeps rod 4 aligned with stem 8. Tool 6 in mounted on rod 4 and used to expand slip 3 as shown in FIG. 8-c. Once expanded, the whole assembly of catheter 1 and tube 5 is pushed forward to remove clip 3 from tissue 2, as shown in FIG. 8-d. At this point clip 3 can be re-installed following the steps in FIG. 2-c to FIG. 2-f or removed completely by pulling clip into tube 5 as shown in FIG. 8-e and FIG. 8-f. Once clip 3 is fully inside tube 5, it can be easily pulled out by hand using rod 4. If desired, clip 3 can be re-used immediately by pushing it back into tube 5 to assume the position shown in FIG. 2-a. When the clip style shown in FIG. 4 is used, the retrieval tool is equipped with a small hook to engage with loop 15.

The large elastic range of Nitinol allows full removal without permanently deforming clip 3. Because of this large elasticity, clip 3 can not be manufactured by cold forming. It has to be held in the relaxed position (shown in FIG. 2-b) and heated to about 510 degrees C. for a few minutes. The exact heat treatment details given by the manufacturer of the Nitinol wire have to be carefully followed.

While the invention will work for any dimension of catheter, the preferred range is for catheters with internal diameters of 4 mm to 15 mm. The Nitinol wire diameter is about 0.4 mm for the 4 mm catheter and about 1 mm for the 15 mm catheter. The thread 10 on stem 8 is from M1 for the 4 mm catheter to M4 on the 15 mm catheter, M2 being a typical value. Tube 5 is made from standard stainless hypodermic tubing. All materials to construct the invention are available from Small Parts Inc (www.smallparts.com).

While the detailed description showed a specific embodiment of a clip with four barbs, it is obvious that the inventions covers many other configurations of barbs, made from many materials including materials used to make absorbable sutures and other non-metallic clips. It is also obvious that the invention can be configured to be used on the outside rather than the inside wall of the body lumen by sliding a clip shown in FIG. 6-b on the outside of tube 5 and expanding it with the method shown in FIG. 7-b.

What is claimed is:

1. A method of closing a hole in a tissue, the method comprising steps of:
inserting an elastic barbed clip via the inside of a tube placed through said hole;
expanding said elastic clip until it covers an area larger than the hole;
attaching said clip to said tissue by embedding said barbs into tissue; and
releasing said clip allowing said barbs to pull tissue towards hole; and
removing said tube.

2. A method of removing a previously installed elastic hole closure clip closing a hole in a tissue, the method comprising the steps of:
inserting a tube through the existing hole in the tissue to bring the tube end into proximity with said clip;
inserting a removal tool via said tube and attaching said tool to said clip;
pulling said clip against the end of said tube proximal to said clip; and
forcing said clip to elastically bend and enter said tube.

3. A hole closure clip sufficiently flexible to be deployed via a catheter to the inner wall of a body lumen and being able to elastically deform from a relaxed state to at least two other states: an expanded state and an installed state, said installed state being larger than the relaxed state but smaller than the expanded state, and said clip being attached to the said inner wall in the expanded state.

4. A method as claimed in claim 1 wherein said barbed clip is made of Nitinol and said expansion is achieved by pulling said clip against the end of said tube.

5. A method as in claim 1 wherein said clip is removably attached to a rod.

6. A method as in claim 1 wherein a test of hole closure is performed before said tube is completely withdrawn from body.

7. A method as in claim 1 wherein said clip can be removed.

8. A method as in claim 1 wherein said clip can be removed using the same tools as used for the installation of the clip.

9. A method as in claim 1 wherein said clip can be detached from said tissue after installation and re-attached.

10. A clip as claimed in claim 3 wherein said clip is made of bent Nitinol wire.

11. A clip as claimed in claim 3 wherein said clip is made of bent Nitinol ribbon.

12. A method as claimed in claim 1 wherein expanding of said clip is done by pulling said clip against end of said tube.

13. A method as claimed in claim 1 wherein expanding of said clip is done by removably attaching a rod to center of said clip and pulling said clip against end of said tube.

14. A method as claimed in claim 2 wherein the clip is re-positioned and re-attached to said tissue instead of being removed through the tube.

15. A clip as in any of the claims above having a threaded portion for the purpose of attachment.
